(19) United States
(12) Patent Application Publication
(10) Pub. No.: US 2024/0335379 A1
(43) Pub. Date: Oct. 10, 2024

(54) STABLE PHARMACEUTICAL COMPOSITIONS OF APOMORPHINE

(71) Applicant: ZYDUS LIFESCIENCES LIMITED, Ahmedabad (IN)

(72) Inventors: Kannan Essakimuthu MUTHAIYYAN, Ahmedabad (IN); Debjani Manoj SINGH, Ahmedabad (IN); Tushar Surajmal NAHATA, Ahmedabad (IN); Abhilash Reddy JONNALA, Ahmedabad (IN); Achuth Devadas SHENOY, Ahmedabad (IN); Darshan Dineshchandra DARJI, Dubai (AE)

(73) Assignee: ZYDUS LIFESCIENCES LIMITED, Ahmedabad (IN)

(21) Appl. No.: 18/293,826

(22) PCT Filed: Aug. 5, 2022
(86) PCT No.: PCT/IB2022/057300

§ 371 Date: Jan. 31, 2024

(30) Foreign Application Priority Data
Aug. 5, 2021 (IN) 202121035398

(51) Int. Cl. A61K 9/08; A61K 9/00; A61K 31/473; A61K 47/02; A61K 47/12; A61K 47/18; A61K 47/20; A61K 47/26

(52) U.S. Cl. CPC A61K 9/08; A61K 9/0019; A61K 31/473; A61K 47/02; A61K 47/12; A61K 47/183; A61K 47/20; A61K 47/26

(57) ABSTRACT

The present invention provides a stable pharmaceutical composition comprising apomorphine and one or more pharmaceutically acceptable carriers in an injectable device. The present invention also provides a process for preparing the stable pharmaceutical composition.

### FIELD OF THE INVENTION

The present invention relates to a stable pharmaceutical composition comprising apomorphine and one or more pharmaceutically acceptable carriers in an injectable device. The present invention also relates to a process for preparing the stable pharmaceutical composition.

### BACKGROUND OF THE INVENTION

Apomorphine hydrochloride is a non-ergoline dopamine agonist. Apomorphine hydrochloride is chemically designated as 6aβ-Aporphine-10,11-diol hydrochloride. It is used as an emetic, as an antidote and for diagnosis and treatment of Parkinson's disease.

Parkinson's disease is a progressive degenerative disease of the central nervous system. Although the primary cause of Parkinson's disease is not known, it is characterized by the degeneration of dopaminergie neurons of the substantia nigra. The degeneration of neurons causes a shortage of dopamine in the brain, which is believed to cause the observable symptoms of the disease. These symptoms include pancity of movement and rigidity, resting tremor, bradykinesia, and poor balance.

In case of the treatment of Parkinson's disease, apomorphine is administered by infusions or by injections subcutaneously. The commercially available product in the United States, APOKYN® is administered subcutaneously and indicated for the acute, intermittent treatment of hypomobility, “off” episodes

International (PCT) Publication No. WO 2015/157212 discloses stable liquid aqueous formulations of apomorphine hydrochloride, which are substantially free of antioxidant agents, such as sodium metabisulfite.

International (PCT) Publication No. WO 2017/055337 discloses aqueous composition of apomorphine, reduced glutathione (GSH) and ascorbic acid, wherein the composition has a pH of about 3 to about 7.4.

International (PCT) Publication No. WO 2013/007381 discloses pharmaceutical composition containing apomorphine, co-solvent, antioxidant, and water having a pH greater than 4.

International (PCT) Publication No. WO 2013/183055 discloses stable liquid or semi-solid pharmaceutical compositions of apomorphine, and an organic acid, which are useful in the treatment of neurological or movement diseases or disorders, e.g., Parkinson's disease, or conditions associated therewith.

International (PCT) Publication No. WO 2016/015875 discloses a composition comprising apomorphine and a divalent metal cation in a molar ratio of 2 or less, and the use of the compositions as a medicament, in particular for treating Parkinson's disease.

A well-known problem of aqueous solutions of apomorphine hydrochloride is stability, since the compound is highly susceptible to oxidation. To improve the stability of apomorphine hydrochloride in an aqueous solution, the use of suitable antioxidants was thought to be essential. However, antioxidant such as sodium metabisulfite can cause injection site reactions like urticarial and restricts product use in hypersensitive individuals. Moreover, benzyl alcohol which is present as a preservative may cause adverse events such as skin necrosis, dermatitis, local site irritation. Injection site reactions, site nodules and skin necrosis are some of the major adverse events, 26% patients reported injection site reactions with Apokyn® in clinical studies.

Moreover, Apokyn® device is a multistep device. It involves loading of cartridge, fixation of needle, priming, dose adjustment etc. before administration. There is a risk of needle injury, dosing errors and also concerns of drug wastage. Specially, Parkinson's disease patient finds this multistep device procedure a bit difficult because of muscle weakness and of lack of dexterity. Bradykinesia and tremors further create concerns for patients to perform multiple steps in device handling.

Thus, there exists a need to develop stable pharmaceutical compositions of apomorphine which can solve above mentioned problems without compromising on the formalation properties.

### SUMMARY OF THE INVENTION

In one general aspect, there is provided a stable pharmaceutical composition comprising apomorphine or a pharmaceutically acceptable salt thereof and one or more pharmaceutically acceptable carriers.

In another aspect, there is provided a stable pharmaceutical composition comprising apomorphine or a pharmaceutically acceptable salt thereof and a stabilizer, an isotonicity agent, or combinations thereof.

In another aspect, there is provided a stable pharmaceutical composition comprising apomorphine or a pharmaceutically acceptable salt thereof and a stabilizer, an isotonicity agent, buffering agents, or combinations thereof.

In another aspect, there is provided a stable pharmaceutical composition comprising apomorphine or a pharmaceutically acceptable salt thereof and a stabilizer, an isotonicity agent. buffering agents, or combinations thereof; wherein the composition has a ph between about 3 and about 4.

In still another aspect, there is provided a stable pharmaceutical composition comprising apomorphine or a pharmacentically acceptable salt thereof, wherein the apomorphine is present as a unit dose in a device.

In further aspect, there is provided a stable pharmaceutical composition comprising apomorphine or a pharmaceutically acceptable salt thereof, wherein the composition does not contain morphine impurity more than 0.5% by weight of spomorphine, as measured by HPLC.

In another aspect, there is provided a method of administering apomorphine comprising: administering a unit dose of apomorphine or a pharmaceutically acceptable salt thereof in a pharmaceutically acceptable carrier subcutaneously to a mammal.

In another aspect, there is provided a stable pharmaceutical composition, wherein the composition is packed under vacuum in a blister packaging comprising an oxygen absorber.

The details of one or more embodiments of the present invention are set forth in the description below. Other features, objects and advantages of the invention will be apparent from the description.

### DETAILED DESCRIPTION OF THE INVENTION

The inventors of the present invention have developed a pharmaceutical composition comprising apomorphine to provide better stability profile and a better patient compliance.

In particular, the present inventors have surprisingly found that the stabilizers in a particular amount provide better stability to the pharmaceutical composition comprising apomorphine without causing reported adverse events. Additionally, isotonicity agent helps to minimize the pain score associated with the subcutaneous administration. The present inventors have also found that the stable composition comprising apomorphine can be administered in a unit dose with a simple device, thus avoiding multiple steps required before administration with typical device available in the market.

The term “apomorpbine” is used in broad sense to include not only the apomorphine per se but also its pharmaceutically acceptable salts, for example apomorphine hydrochloride.

The term “pharmaceutically acceptable salts” refers to those salts which retain the biological effectiveness and properties of the free bases or free acids, which are not biologically or otherwise undesirable.

The term “pharmaceutically acceptable carrier” as used herein, means a pharmaceutically acceptable material, composition, or vehicle, such as a liquid or solid filler, excipient, or a solvent.

The term “pharmaceutical composition” as used herein, means a product comprising an active compound or a salt thereof together with pharmaceutical excipients such as stabilizers, buffers, antioxidants, and tonicity modifiers, said pharmaceutical composition being useful for treating. preventing or reducing the severity of a disease or disorder by administration of said pharmaceutical composition to a person.

The term, “about” means plus or minus approximately ten percent of the indicated value.

The term “effective amount” as used herein, means a dosage which is sufficient in order for the treatment of the patient to be effective compared with no treatment.

The term “stabilizer” as used herein, means a compound which inhibits or prevents the degradation of apomorphine hydrochloride so that it can be used in a pharmaceutical formulation while retaining much of its potency.

The term “isotonicity agent” as used refers to a chemical compound in a pharmaceutical composition that serves to modify the osmotic pressure of the pharmaceutical composition, so that the osmotic pressure becomes closer to that of human plasma.

The term “stable” as used herein, refers to any composition comprising a drug having sufficient physical and chemical stability to allow storage at a convenient temperature, such as between about 0° C. and about 40° C., for a commercially reasonable period of time.

In one aspect, there is provided a stable pharmaceutical composition comprising apomorphine or a pharmaceutically acceptable salt thereof and one or more pharmaceutically acceptable carriers.

In another aspect, there is provided a stable pharmaceutical composition comprising apomorphine or a pharmaceutically acceptable salt thereof and a stabilizer, an isotonicity agent. or combinations thereof.

In one embodiment, the stable pharmaceutical composition comprises apomorphine in an amount of from about 1.5 mg/mL to about 10 mg/mL, for example about 1.66 mg/mL, about 3.33 mg/mL, about 5 mg/mL, about 6.66 mg/mL, about 8.33 mg/mL or about 10 mg/mL.

In one aspect, there is provided a stable pharmaceutical composition comprising apomorphine or a pharmaceutically acceptable salt thereof, and a pharmaceutically acceptable carrier, wherein the apomorphine is present as a unit dose in a device.

In one embodiment, the stable pharmaceutical composition of apomorphine in unit dose is provided in a device such as a prefilled syringe (PFS), pen or an autoinjector.

In one embodiment, the stable pharmaceutical composition of apomorphine in unit dose is provided in an autoinjector.

In another embodiment, the stable pharmaceutical composition comprising apomorphine may have a pH of from about 1 to about 4, for example, about 1, about 1.5, about 2, about 2.5, about 3. about 3.5. or about 4.

The suitable pharmaceutically acceptable carriers of the present invention may include one or more pharmaceutically acceptable stabilizers, isotonicity agents, antioxidants, surfactants, buffering agents, solvents, and pH adjusting agents.

Suitable stabilizers include, but are not limited to, ethylenediaminetetraacetic acid (EDTA), disodium EDTA, sodium EDTA, calcium disodium EDTA,

hydroxyethylethylenediaminetriacetic acid (HEDTA), docosatetraenoic acid (DTA), nitrilotriacetic acid (NTA), hydroxyaminopolycarboxylic acid (HACA), diethylenetriaminepentaacetic acid (DTPA), hydroxyethyliminodiacetic acid (HEIDA), alcohol and the like, or combinations thereof.

The stabilizers may be present in an amount of from about 0.1 mg/mL to about 10 mg/mL.

In one embodiment, the stabilizer comprises EDTA in an amount of from about 0.1 mg/mL to about 2 mg/mL, for example about 0.1 mg/mL, about 0.5 mg/mL, about 1 mg/mL, about 1.5 mg/mL, or about 2 mg/mL.

Suitable antioxidants include, but are not limited to, sodium formaldehyde sulfoxylate, monothioglycerol, ascorbic acid, fumaric acid, citric acid, malic acid, gallic acid and its salts and esters, sodium metabisulphite, sodium sulphite, potassium metabisulphite, methionine, dithionite sodium (sodium hydrosulfite, sodium sulfoxylate), BHA (butylated hydroxyanisole), BHT (butylated hydroxytoluene), vitamin E, vitamin E acetate, vitamin E PEG 1000 succinate and the like or combinations thereof.

The antioxidants may be present in an amount of from about 0.1 mg/mL to about 5 mg/mL,

In one embodiment, the antioxidant comprises sodium formaldehyde sulfoxylate, monothioglycerol or a combination thereof in an amount of from about 0.1 mg/mL to about 5 mg/mL.

In another embodiment, the antioxidant comprises sodium formaldehyde sulfoxylate in an amount of from about 0.1 mg/mL, to about 3 mg/mL, for example about 0.1 mg/mL, about 0.5 mg/mL, about 0.7 mg/mL, about 1 mg/mL, about 1.5 mg/mL, about 2 mg/mL, about 2.5 mg/mL, or about 3 mg/mL. The sodium formaldehyde sulfoxylate may be present in an anhydrous or a dihydrate form.

In another embodiment, the antioxidant comprises monothioglycerol in an amount of from about 0.1 mg/mL to about 5 mg/mL, for example about 0.1 mg/mL, about 0.5 mg/mL, about 0.7 mg/mL, about 1 mg/mL, about 1.5 mg/mL, about 2 mg/mL, about 2.5 mg/ml, about 3 mg/mL, about 3.5 mg/mL, about 4 mg/mL, about 4.5 mg/mL, or about 5 mg/mL.

Suitable isotonicity agents include, but are not limited to, sodium chloride, potassium chloride, calcium chloride, magnesium chloride, glucose, sucrose, dextrose, mannitol, sorbitol, glycerol, trehalose, propylene glycol or any combinations thereof.

In one embodiment, the isotonicity agent comprises mannitol, sorbitol, or combination thereof in an amount of from about 20 mg/mL to about 100 mg/mL, for example, about 20 mg/mL to about 80 mg/mL, about 30 mg/mL to about 70 mg/mL, or about 40 mg/mL to about 50 mg/ml.

In one embodiment, the isotonicity agent comprises sodium chloride in an amount of from about 1 mg/mL to about 20 mg/mL, for example, about 2 mg/mL to about 18 mg/mL, about 4 mg/mL to about 16 mg/mL, or about 8 mg/mL to about 14 mg/mL.

Suitable pharmaceutically acceptable pH adjusting agents include, but not limited to, sodium hydroxide, hydrochloric acid, boric acid, citric acid, acetic acid, phosphoric acid, succinic acid, potassium hydroxide, ammonium hydroxide, magnesium oxide, calcium carbonate, magnesium carbonate, malic acid, potassium citrate, sodium phosphate, lactic acid, gluconic acid, tartaric acid, fumaric acid, diethanolamine, monoethanolamine, sodium carbonate, sodium bicarbonate, triethanolamine, or any combinations thereof.

In one embodiment, the pH adjusting agent comprises hydrochloric acid in an amount to provide pH of the solution between about 1 and about 4, for example, about 1, about 2, about 3, or about 4.

Suitable pharmaceutically acceptable buffering agents include, but not limited to, acetate buffer (e.g., sodium acetate), citrate buffer (e.g., citric acid/sodium citrate), phosphate buffer (e.g., monobasic sodium phosphate, dibasic sodium phosphate etc.), carbonate buffer. HCl buffer, phthalate buffer, glycine-HCl buffer, or any combinations thereof.

In one embodiment, the buffering agent comprises sodium dihydrogen phosphate monohydrate, citric acid, sodium hydroxide or any combinations thereof in an amount sufficient to provide pH of the solution between about 1 and about 4.

Suitable pharmaceutically acceptable preservatives include, but not limited to, chlorobutanol, benzalkonium chloride, methyl paraben, propyl paraben, benzoic acid, sodium benzoate, sorbic acid, henzethonium chloride, cetyl pyridinium chloride, benzyl bromide, benzyl alenhol, phenylmercury nitrate, phenylmercury acetate, thiomersal, merthiolate, chlorhexidine, phenylethyl alcohol, quaternary ammonium chloride, sodium benzoate, sodium propionate, or any combinations thereof.

In one embodiment, the stable pharmaceutical composition of apomorphine is substantially free of any preservative.

In another embodiment, the stable pharmaceutical composition comprising apomorphine does not contain total impurity more than 3%, for example, not more than 2.5%, not more than 2.09%, not more than 1.5%, or not more than 1.0% by weight of apomorphine, as measured by HPLC.

In another embodiment, the stable pharmaceutical composition comprising apomorphine does not contain morphine impurity more than 0.5%, for example, not more than 0.4%, not more than 0.39%, not more than 0.2%, or not more than 0.1% by weight of apomorphine, as measured by HPLC.

In another embodiment, the stable pharmaceutical composition comprising apomorphine docs not contain orthoquinone impurity more than 75 ppm, for example, not more than 50 ppm, not more than 30 ppm, not more than 10 ppm, or not detected, as measured by HPLC.

In another embodiment, the stable pharmaceutical composition comprising apomorphine does not contain unspecified impurity more than 0.5%, for example, not more than 0.4%, not more than 0.3%, not more than 0.2%, or not more than 0.19% by weight of apomorphine, as measured by HPLC.

In one embodiment, the stable pharmaceutical composition comprising apomorphine retains at least about 90% of the potency of apomorphine, for example at least about 93%, at least about 95%, at least about 97% or at least about 99% of the potency after storing at 40° C. and 75% relative humidity for at least three months.

In one embodiment, the stable pharmaceutical composition comprising apomorphine has an osmolality of about 150-350 mOsm/kg, for example, about 200-350 mOsm/kg, or about 270-340 mOsm/kg.

According to one embodiment, the stable pharmaceutical composition uses inert gas to minimize oxidation of the sensitive material. This may be aided by, for example, purging the sealable container with a gas which is substantially oxygen-free, or substantially moisture free. The gas used for purging the scalable container may be any appropriate inert gas known to those in the art, the most used gases being argon, helium, carbon dioxide or nitrogen, or combinations thereof. In another embodiment, the inert gas is nitrogen or carbon dioxide.

In one embodiment, there is provided a method of administering apomorphine comprising: administering a unit dose of apomorphine or a pharmaceutically acceptable salt thereof in a pharmaceutically acceptable carrier subcutaneously to a mammal, wherein the unit dose is administered with the help of an autoinjector.

According to one embodiment, the stable pharmaceutical composition comprises;
(i) apomorphine in an amount of from about 1.5 mg/mL to about 10 mg/mL;
(ii) one or more stabilizers in an amount of from about 0.1 mg/mL to about 10 mg/mL;
(iii) one or more pharmaceutically acceptable carriers: wherein the apomorphine is present as a unit dose in a device.

According to one embodiment, the stable pharmaceutical composition comprises:
(i) apomorphine in an amount of from about 1.5 mg/mL to about 10 mg/mL;
(ii) EDTA in an amount of from about 0.5 mg/mL to about 1 mg/mL;
(iii) one or more pharmaceutically acceptable carriers: wherein the composition has a pH of from about 3 to about 4.

According to one embodiment, the stable pharmaceutical composition comprises:
(i) apomorphine in an amount of from about 1.5 mg/mL to about 10 mg/mL;
(ii) mannitol in an amount of about 45 mg/mL;
(iii) one or more pharmaceutically acceptable carriers; wherein the composition has a pH of from about 3 to about 4.

According to one embodiment, the stable pharmaceutical composition comprises:
(i) apomorphine in an amount of from about 1.5 mg/mL to about 10 mg/mL;
(ii) EDTA in an amount of from about 0.5 mg/mL to about 1 mg/mL;
(iii) mannitol in an amount of about 45 mg/mL;
(iv) one or more pharmaceutically acceptable carriers; wherein the composition has a pH of from about 3 to about 4.

According to one embodiment, the stable pharmaceutical composition comprises:
(i) apomorphine in an amount of from about 1.5 mg/mL to about 10 mg/mL;
(ii) EDTA in an amount of from about 0.5 mg/mL to about 1 mg/mL;
(iii) mannitol in an amount of about 45 mg/mL;
(iv) sodium formaldehyde sulfoxylate in an amount of 0.7 mg/mL;
(v) one or more buffering agents: wherein the composition has a pH of from about 3 to about 4.

According to one embodiment, the stable pharmaceutical composition comprises:
(i) apomorphine in an amount of from about 1.5 mg/mL to about 10 mg/mL;
(ii) EDTA in an amount of from about 0.52 mg/mL;
(iii) mannitol in an amount of about 40 mg/mL;
(iv) sodium formaldehyde sulfoxylate in an amount of 1.0 mg/mL; and
(v) a combination of sodium dibydrogen phosphate monohydrate and citric acid (anhydrous), wherein the composition has a pH of from about 2.5 to about 4.

According to one embodiment, the stable pharmaceutical composition comprises:
(i) apomorphine in an amount of from about 1.5 mg/mL to about 10 mg/mL;
(ii) EDTA in an amount of from about 0.5 mg/mL to about 1 mg/mL;
(iii) mannitol in an amount of about 45 mg/mL;
(iv) sodium formaldehyde sulfoxylate, monothieglycerol or combination thereof in an amount of from about 0.5 mg/mL to about 1.5 mg/mL; and
(v) one or more buffering agents, wherein the composition has a pH of from about 3 to about 4.

1.5

According to one embodiment, the stable pharmaceutical composition comprises:
(i) apomorphine in an amount of from about 1.5 mg/mL to about 10 mg/mL;
(ii) EDTA in an amount of from about 0.5 mg/mL to about 1 mg/mL;
(iii) mannitol in an amount of about 45 mg/mL; and
(iv) one or more pharmaceutically acceptable carriers, wherein the apomorphine is present as a unit dose in a device.

As a further embodiment, the invention provides a process for preparing the stable pharmaceutical composition of apomorphine comprising the following steps:
(a) sparging water for injection with nitrogen or carbon dioxide,
(b) adding and dissolving stabilizer, antioxidant, isotonicity agent into the water of step (a),
(c) adding pH adjusting agent to adjust pH of the solution between about 3 and about 4,
(d) adding apomorphine to step (c),
(e) optionally adjusting the pH between about 3 and about 4,
(f) making up the final solution with water for injection and sparging with nitrogen or carbon dioxide,
(g) filtering and filling the solution in PFS, and
(h) assembling the PES into an autoinjector.

As a further embodiment, the invention provides a process for preparing the stable pharmaceutical composition of apomorphine comprising the following steps:
(a) sparging water for injection with nitrogen,
(b) adding and dissolving stabilizer, antioxidant, isotonicity agent into the water of step (a),
(c) adding buffer to maintain pH of the solution between about 3 and about 4,
(d) adding apomorphine to step (e),
(e) optionally adjusting the pH between about 3 and about 4,
(f) making up the final solution with water for injection and sparging with nitrogen,
(g) filtering and filling the solution in PFS, and
(h) assembling the PES into an autoinjector.

As a further embodiment, the invention provides a process for preparing the stable pharmaceutical composition of apomorphine comprising the following steps:
(a) sparging water for injection with nitrogen,
(b) adding and dissolving stabilizer, isotonicity agent into the water of step (a),
(c) adding buffer to maintain pH of the solution between about 3 and about 4,
(d) adding antioxidant followed by apomorphine to step (c),
(c) optionally adjusting the pH between about 3 and about 4,
(f) making up the final solution with water for injection and sparging with nitrogen,
(g) filtering and filling the solution in PES, and
(h) assembling the PFS into an autoinjector.

As a further embodiment, the invention provides a process for preparing the stable pharmaceutical composition of apomorphine comprising the following steps:
(a) sparging water for injection with nitrogen,
(b) adding and dissolving mannitol, sodium formaldehyde sulfoxylate, monothioglycerol into the water of step (a),
(c) adding buffer to maintain pH of the solution between about 3 and about 4,
(d) adding apomorphine to step (c),
(e) optionally adjusting the pH between about 3 and about 4,
(f) making up the final solution with water for injection and sparging with nitrogen,
(g) filtering and filling the solution in PFS, and
(h) assembling the PFS into an autoinjector.

In one embodiment, a prefilled syringe or an autoinjector is packed under vacuum along with an oxygen absorber and/or an oxygen level indicator. In another embodiment, the invention provides a secondary packaging system for injectable apomorphine, the packaging system comprising a prefilled syringe filled under inert conditions with apomorphine, a hermetically sealed blister packaging which covers the prefilled syringe, wherein the blister packaging optionally comprises an oxygen absorber, wherein the oxygen absorber reduces the oxygen level from the time of packaging assembly to about zero percent. In some embodiments, the oxygen absorber is in the form of a canister and is selected from ascorbic acid, sodium ascorbate, reduced iron compounds like iron hydroxide, iron oxide etc., catechol, phenol, activated carbon, other metal ligands etc. In some embodiments, prefilled syringe is assembled into an autoinjector, which is ultimately packed in the above-mentioned secondary packaging system.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1
Pharmaceutical composition of apomorphine

| Sr. | | Quantity |
|---|---|---|
| No. | Ingredient | (mg/mL) |
| 1 | Apomorphine HCl | 3.33 |
| 2 | Sodium formaldehyde sulfoxylate | 0.71 |
| 3 | Mannitol | 45 |
| 4 | EDTA | 1.0 |
| 5 | HCl/NaOH | q.s. |
| 6 | Water for injection | q.s. to 1 mL |

### Procedure

(a) water for injection was sparged with nitrogen,
(b) mannitol, sodium formaldehyde sulfoxylate. EDTA were added sequentially and dissolved into the water of step (a),
(c) pH adjusting agent like HCl or NaOH was added to have pH of the solution between about 3 and about 4,
(d) apomorphine was added and dissolved into step (c) solution,
(e) pH was adjusted between about 3 and about 4,
(f) final solution was made up with water for injection and sparged with nitrogen,
(g) solution obtained in step (f) was filtered and filled in PFS, and
(h) the PFS was assembled into an autoinjector.

### Example 2

TABLE 2
Pharmaceutical composition of apomorphine

| Sr. | | Quantity |
|---|---|---|
| No. | Ingredient | (mg/mL) |
| 1 | Apomorphine HCl | 5 |
| 2 | Sodium formaldehyde sulfoxylate | 0.71 |
| 3 | Mannitol | 45 |
| 4 | EDTA | 1.0 |
| 5 | Sodium dihydrogen phosphate monohydrate | q.s. |
| 6 | Citric acid (anhydrous) | q.s. |
| 7 | HCl/NaOH | q.s. |
| 8 | Water for injection | q.s. to 1 mL |

### Procedure

(a) water for injection was sparged with nitrogen,
(b) mannitol, sodium formaldehyde sulfoxylate, EDTA were added sequentially and dissolved into the water of step (a),
(c) pH of the solution was checked, and buffer was added gradually to adjust the pH between about 3 and about 4,
(d) apomorphine was added and dissolved into step (c) solution,
(e) pH was adjusted between about 3 and about 4,
(f) final solution was made up with water for injection and sparged with nitrogen,
(g) solution obtained in step (f) was filtered and filled in PFS, and
(h) the PFS was assembled into an autoinjector.

### Example 3

TABLE 3
Pharmaceutical composition of apomorphine

| Sr. | | Quantity |
|---|---|---|
| No. | Ingredient | (mg/mL) |
| 1 | Apomorphine HCl | 6.66 |
| 2 | Sodium formaldehyde sulfoxylate | 0.71 |
| 3 | Monothioglycerol | 1.19 |
| 4 | Mannitol | 45 |
| 5 | Sodium dihydrogen phosphate monohydrate | q.s. |
| 6 | Citric acid (anhydrous) | q.s. |
| 7 | HCl/NaOH | q.s. |
| 8 | Water for injection | q.s. to 1 mL |

### Procedure

(a) water for injection was sparged with nitrogen,
(b) mannitol, sodium formaldehyde sulfoxylate, monothioglycerol were added sequentially and dissolved into the water of step (a),
(c) pH of the solution was checked, and buffer was added gradually to adjust the pH between about 3 and about 4,
(d) apomorphine was added and dissolved into step (c) solution,
(e) pH was adjusted between about 3 and about 4,
(f) final solution was made up with water for injection and sparged with nitrogen,
(g) solution obtained in step (f) was filtered and filled in PFS, and
(h) the PAS was assembled into an autoinjector.

### Example 4

TABLE 4
Pharmaceutical composition of apomorphine

| Sr. | | Quantity |
|---|---|---|
| No. | Ingredient | (mg/mL) |
| 1 | Apomorphine HCl | 10 |
| 2 | Sodium formaldehyde sulfoxylate | 0.71 |
| 3 | Monothioglycerol | 1.19 |
| 4 | Mannitol | 45 |
| 5 | EDTA | 1.0 |
| 6 | Sodium dihydrogen phosphate monohydrate | q.s. |
| 7 | Citric acid (anhydrous) | q.s. |
| 8 | HCl/NaOH | q.s. |
| 9 | Water for injection | q.s. to 1 mL |

### Procedure

(a) water for injection was sparged with nitrogen,
(b) mannitol, sodium formaldehyde sulfoxylate, monothioglycerol and EDTA were added sequentially and dissolved into the water of step (a),
(c) pH of the solution was checked, and buffer was added gradually to adjust the pH between about 3 and about 4,
(d) apomorphine was added and dissolved to step (c) solution,
(e) pH was adjusted between about 3 and about 4,
(f) final solution was made up with water for injection and sparged with nitrogen,
(g) solution obtained in step (f) was filtered and filled in PFS, and
(h) the PFS was assembled into an autoinjector.

### Example 5

TABLE 5
Pharmaceutical composition of apomorphine

| Sr. | | Quantity |
|---|---|---|
| No. | Ingredient | (mg/mL) |
| 1 | Apomorphine HCl (as hemihydrate) | 3.33 mg/mL |
| 2 | Sodium formaldehyde sulfoxylate (as dihydrate) | 1.3 |
| 3 | Mannitol | 40 |
| 4 | EDTA | 0.52 |
| 5 | Sodium dihydrogen phosphate monohydrate | 0.46 |
| 6 | Citric acid (anhydrous) | 0.80 |
| 7 | HCl/NaOH | q.s. |
| 8 | Water for injection | q.s. to 1 mL |

### Procedure

(a) water for injection was sparged with nitrogen,
(b) mannitol and EDTA were added sequentially and dissolved into the water of step (a),
(c) pH of the solution was checked, and buffer (combination of sodium dihydrogen phosphate monohydrate and citric acid) was added gradually to adjust the pH between about 3 and about 4,
(d) sodium formaldehyde sulfoxylate was added followed by the addition of apomorphine and dissolved into step (c) solution,
(e) pH was adjusted by NaOH or HCl between about 3 and about 4,
(f) final solution was made up with water for injection and sparged with nitrogen,
(g) solution obtained in step (f) was filtered and filled in PFS, and
(h) the PFS was assembled into an autoinjector.

The autoinjectors were packed in a blister pack under various conditions such as temperature and humidity for some months, and were checked for various parameters such as clarity of the solution, pH, osmolality of the solution, assay of apomorphine, impurity levels etc. for determining stability of the batch. The results are shown in the table below.

| | | | | with nitrogen overlay + oxygen | | | | | |
|---|---|---|---|---|---|---|---|---|---|
| | with oxygen buster | | | buster | | | with nitrogen overlay | | |
| | | 1 M | 1 M | | 1 M | 1 M | | 1 M | 1 M |
| | | 25 ± 2° C./ | 40 ± 2° C./ | | 25 ± 2° C./ | 40 ± 2° C./ | | 25 ± 2° C./ | 40 ± 2° C./ |
| Test | Initial | 60 ± 5% RH | 75 ± 5% RH | Initial | 60 ± 5% RH | 75 ± 5% RH | Initial | 60 ± 5% RH | 75 ± 5% RH |
| Description | Clear | Clear | Clear | Clear | Clear | Clear | Clear | Clear | Clear |
| of solution | | | | | | | | | |
| pH | 3.06 | 3.22 | 3.32 | 3.10 | 3.38 | 3.40 | 3.08 | 3.38 | 3.42 |
| Osmolality | 281 | 283 | 281 | 278 | 281 | 283 | 280 | 286 | 284 |
| Assay of | 99.1% | 98.70% | 100.0% | 99.6% | 98.40% | 98.80% | 100.1 | 100.60% | 100.60% |
| Apomorphine | | | | | | | | | |
| HCl | | | | | | | | | |
| Orthoquinone | ND | ND | ND | ND | ND | ND | ND | ND | ND |
| Impurity | | | | | | | | | |
| Morphine | ND | ND | ND | ND | ND | ND | ND | ND | ND |
| Impurity | | | | | | | | | |
| Any | ND | ND | ND | ND | ND | ND | ND | ND | ND |
| unspecified | | | | | | | | | |
| degradant | | | | | | | | | |
| Total | 0.07 | 0.06 | 0.06 | 0.07 | 0.06 | 0.06 | 0.07 | 0.06 | 0.06 |
| degradation | | | | | | | | | |
| products | | | | | | | | | |

Some of the autoinjectors were packed in an aluminum pouch, with vacuum and oxygen buster under various conditions such as temperature and humidity for some months, and were checked for various parameters such as clarity of the solution, pH, osmolality of the solution, assay of apomorphine, impurity levels etc. for determining stability of the batch. The results are shown in the table below.

| | | 1 M | 3 M | 6 M | 1 M | 3 M | 6 M |
|---|---|---|---|---|---|---|---|
| | | 25 ± 2° C./ | 25± 2° C./ | 25 ± 2° C./ | 40 ± 2° C./ | 40 ± 2° C./ | 40 ± 2° C./ |
| Test | Initial | 60 ± 5% RH | 60 ± 5% RH | 60 ± 5% RH | 75 ± 5% RH | 75 ± 5% RH | 75 ± 5% RH |
| Description of solution | Clear | Clear | Clear | Clear | Clear | Clear | Clear |
| pH | 3.23 | 3.24 | 3.21 | 3.27 | 3.23 | 3.22 | 3.44 |
| Osmotality | 193 | 192 | 194 | 199 | 193 | 194 | 194 |
| Assay of Apomorphine HCl | 99.8% | 100.9% | 100.4% | 100.5% | 101.0% | 100.6% | 101.3% |
| Orthoquinone Impurity | ND | ND | ND | ND | ND | ND | ND |
| Morphine Impurity | ND | ND | ND | BQL | ND | ND | BQL |
| Apocodeine Impurity | BQL | BQL | BQL | ND | BQL | BQL | ND |
| Pseudo morphine | ND | ND | ND | BQL | ND | ND | BQL |
| Impurity | | | | | | | |
| Any unspecified | ND | ND | ND | BQL | ND | ND | BQL |
| degradant | | | | | | | |
| Total degradation | 0.0% | 0.0% | 0.0% | 0.0% | 0.0% | 0.0% | 0.0% |
| products | | | | | | | |

ND: Not Detected
BQL: Below Quantitation Limit (0.05%)

It is clear from the above stability results that the composition remains stable for longer period if packed in various packing configurations under vacuum.

1. A stable pharmaceutical composition comprising apomorphine or a pharmaceutically acceptable salt thereof, and a pharmaceutically acceptable carrier, wherein the apomorphine is present as a unit dose in a device.

2. The stable pharmaceutical composition according to claim 1, wherein the device is a prefilled syringe, pen, or an autoinjector.

3. The stable pharmaceutical composition according to claim 2, wherein the device is an autoinjector.

4. The stable pharmaceutical composition according to claim 1, wherein the apomorphine is present in a concentration of from about 1.5 mg/ml to about 10 mg/ml.

5. The stable pharmaceutical composition according to claim 4, wherein the apomorphine is present in the concentration of about 1.66 mg/ml, about 3.33 mg/ml, about 5 mg/ml, about 6.66 mg/ml, about 8.33 mg/ml, or about 10 mg/ml.

6. The stable pharmaceutical composition according to claim 1, wherein the composition is suitable for subcutaneous administration.

7. The stable pharmaceutical composition according to claim 1, wherein the composition further comprises one or more antioxidants.

8. The stable pharmaceutical composition according to claim 7, wherein the antioxidant comprises sodium formaldehyde sulfoxylate, monothioglycerol, or a combination thereof.

9. The stable pharmaceutical composition according to claim 1, wherein the composition further comprises one or more stabilizers.

10. The stable pharmaceutical composition according to claim 9, wherein the stabilizer is EDTA.

11. The stable pharmaceutical composition according to claim 1, wherein the composition further comprises one or more buffering agents.

12. The stable pharmaceutical composition according to claim 11, wherein the buffering agent comprises acetate buffer, citrate buffer, phosphate buffer, or combinations thereof.

13. The stable pharmaceutical composition according to claim 1, wherein the composition further comprises one or more isotonicity agents.

14. The stable pharmaceutical composition according to claim 13, wherein the isotonicity agent comprises mannitol, sorbitol, sodium chloride, or combinations thereof.

15. The stable pharmaceutical composition according to claim 1, wherein the composition is substantially free of preservatives.

16. The stable pharmaceutical composition according to claim 1, wherein the composition has a pH of between about 3.0 and about 4.0.

17. A method of administering apomorphine comprising administering a unit dose of apomorphine or a pharmaceutically acceptable salt thereof in a pharmaceutically acceptable carrier subcutaneously to a mammal, wherein the unit dose is administered with the help of an autoinjector.

18. The stable pharmaceutical composition according to claim 1, wherein the composition is packed under vacuum in a blister packaging comprising an oxygen absorber.

19. The stable pharmaceutical composition according to claim 1, wherein the composition is packed in an aluminum pouch under vacuum.

20. A stable pharmaceutical composition comprising apomorphine or a pharmaceutically acceptable salt thereof, and a pharmaceutically acceptable carrier, wherein the composition does not contain morphine impurity more than 0.5% by weight of apomorphine, as measured by HPLC.
